(19) United States
(12) Patent Application Publication
(10) Pub. No.: US 2023/0225702 A1
(43) Pub. Date: Jul. 20, 2023

(54) Real-time image analysis for vessel detection and blood flow differentiation

(71) Applicant: Telemed, UAB, Vilnius (LT)

(72) Inventor: Andrius Sakalauskas, Vilnius (LT)

(21) Appl. No.: 17/575,663

(22) Filed: Jan. 14, 2022

(51) Int. Cl. A61B 8/08; A61B 8/06; G06N 3/08; G16H 30/40

(52) U.S. Cl. CPC A61B 8/085; A61B 8/5246; A61B 8/06; G06N 3/08; G16H 30/40

(57) ABSTRACT

This invention discloses an image analysis system and method, which detects blood vessels in ultrasound structural B-mode images using deep learning and identifies blood vessel type (vein or artery) based on automatic Doppler spectrogram features analysis. Such an automatic solution is important for successful catheter insertion under ultrasound guidance or other procedures which requires differentiation between the arteries or the veins or quantitative characterization of blood flow. The system contains: an ultrasound scanner with implemented B-mode and PW mode equipped with probe and algorithms implemented as software modules in the ultrasound scanner: 1) for automatic vessel tracking in real-time based on deep learning and, 2) algorithms for Doppler spectrogram quality assessment and parameterization by using quantitative spectrogram features. The system detects and classifies scanned vessels according to blood flow into: 1) arteries, or 2) veins.

### FIELD OF INVENTION

The present invention relates to medical image analysis in general, and in particular, to ultrasound image analysis. The invention further teaches the application of multiple modes of machine learning and deep learning for image analysis.

### BACKGROUND OF THE INVENTION

In the field of medical imaging, image analysis has moved from the speciality of trained technicians to specialized machine learning algorithms. Trained machine learning algorithms can reliably and accurately solve general image recognition or pattern recognition problems. In complex medical situations, such as with multi-mode imaging diagnostics, image analysis becomes more complex because multiple types of images need to be evaluated.

Ultrasound medical imaging systems consist of numerous different imaging modes. The present invention teaches automatization and analysis of two in particular—brightness mode (B-mode) and spectral Doppler mode (pulsed wave (PW))—which are commonly used alone or in conjunction for diagnostic and procedural medical applications. B-mode ultrasound provides two-dimensional images of the magnitude of the reflections in tissues and represents structural information. Meanwhile, spectral Doppler imaging mode is used for representation of fluid flow (such as blood flow) information from a defined location, called Doppler gates.

It is standard in the field of vein cannulation to use ultrasound to guide catheter insertion, among other applications of ultrasound imaging. A standard procedure of cannulation requires a technician to scan for blood vessels, manually identify blood vessels, and then the technician can switch ultrasound modes of operations and manually adjust the imaging parameters. Ultimately, ultrasound scanning provides some information to the technician about the state of the vessels and their character, and while numerous known solutions are provided to determine the character of a certain type of vessel or under specific conditions, reliable means of identification of an artery or vein in general are not found in the art. Most commonly, ultrasound scanners used for vein cannulation guidance are capable of only B-mode ultrasound scanning, and therefore, lack the capability for more precise diagnostic functionality.

A trend in the field of medical imaging is to automate the role of a radiological technician by applying image-recognition software solutions. Such solutions have been developed for various medical imaging mediums, including specific vessel detection, such as carotid artery or jugular vein, in B-mode images, however, these solutions are parameterized for only structural 2D images and cannot be applied to vessel classification in general. Error in proper identification of artery and veins can be fatal; in the case of puncturing the jugular vein, it could be confused with carotid artery and that might result into severe complications.

The present invention improves on state-of-the-art solutions for automation of medical image analysis, specifically, classification of blood vessels using a combination of B-mode and spectral Doppler ultrasound imaging for any blood vessel. In standard procedures, multiple steps require manual manipulation or analysis by a trained technician, and in the present invention, all of the manual steps are replaced by machine learning algorithms Comparison of the steps that are automated in the present invention will become apparent in the below description.

### SUMMARY OF INVENTION

A system and a method hereby described are intended to detect blood vessels by applying trained deep learning (DL) algorithms to ultrasound structural B-mode images and subsequently to identity vessel character (vein or artery) using Doppler spectrogram image analysis by trained machine learning (ML) classification models. The DL detection in B-mode images is followed by automatic positioning of PW Doppler gates followed by PW Doppler imaging scans. The resulting Doppler images of identified blood vessels are used for further classification of the vessels as either vein or artery using either spectrogram feature extraction combined with machine learning predictions or image recognition using deep learning predictions. Such classification is important for successful catheter insertion (cannulation) under ultrasound guidance or other procedures which requires differentiation between arteries and veins or quantitative characterization of blood flow. Previous studies have shown that usage of PW Doppler during vein cannulation increases first pass success rate.

The proposed system allows fully automated detection of vessels and vessel type differentiation and does not require manual Doppler gate placement, and therefore there is no need for a highly qualified technician with deep knowledge of spectral Doppler to perform the procedure. The presented solution is based on machine learning principles. It works in real-time with B-mode imaging having a frame rate close to 50 frames/second. Deep leaning-based detection is highly accurate compared with known object detection techniques. The differentiation of arteries and veins is typically done by evaluating blood flow velocity, but this feature alone is not sufficient for accurate classification, because in the diastolic periods blood flow velocity is comparable for arteries and veins. Herein, it is taught that a PW Doppler spectrogram of a blood vessel contains more features that can be used to more accurately classify a blood vessel.

The image analysis system consists of an ultrasound scanner, an array probe capable of performing B-mode and PW mode scanning, and one or more computer processors configured with computer-implemented methods 1) for automatic vessel tracking in real-time based on DL, 2) for Doppler spectrogram quality assessment, and 3) for classification of a detected vessel as an artery or a vein based on machine learning models.

### BRIEF DESCRIPTION OF THE DRAWINGS

The invention can be best understood by referring to the drawings, which depict preferred embodiments of the present invention.

FIG. 1 is a schematic block diagram of the preferred embodiment of the image analysis system for automatic vessel detection and blood flow type differentiation as it can be preferably employed in an ultrasound scanner.

FIG. 2 is a flowchart of the method for image analysis and blood flow differentiation (arterial/venous).

FIG. 3 is a sketch of typical convolutional neural network architecture with base components used for blood vessel detection.

FIG. 4 is a diagram for the spectral Doppler beam control and PW data analysis over time.

FIG. 5 is an illustration of spectrogram binarization procedure to separate blood flow related pixels from background noise.

FIG. 6 is an example of data representation in a minimalistic graphical user interface, which presents the outcome of the system (vessel detection and classification).

The presented figures are for illustration and the scale, the proportions, and the other aspects do not necessarily correspond to the actual technical solution.

### DETAILED DESCRIPTION OF THE INVENTION

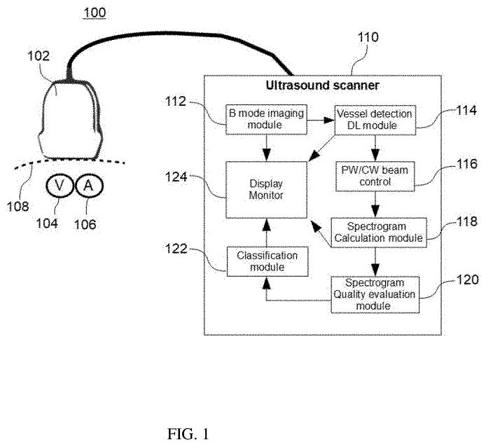

The present invention is best described by its preferred embodiments, which are exemplified by the figures. According to the schematic block diagram of FIG. 1, the image analysis system 100 for vessel detection and vessel type differentiation functions by use of an ultrasound probe 102 used externally on the tissue surface 108. The system contains an ultrasound scanner 110 equipped with the ultrasound probe 102. The ultrasound probe can be linear, convex, or phased array depending on the intended use and are driven by a high voltage pulser, which excites the probe's piezo elements to transmit acoustic waves into the tissue. In principle, the B-mode and the spectral Doppler mode excitation voltage sequences differs by the number of pulse periods and pulse repetition frequency, wherein shorter sequences for B-mode imaging and longer sequences for spectral Doppler with high pulse repetition frequency are used. The ultrasound scanner 110 either contains hardware for data storage and data processing such as one or more computer processors and/or is connected to a personal computer (PC) capable of data storage and date processing. In a preferred embodiment, all data collected from the probe is processed by software modules that are programmatically embedded in the scanner hardware, on the connected PC, and/or embedded on a computer program product being tangibly embodied on a non-transitory computer-readable medium and comprising executable code. The implemented software modules include B-mode imaging module 112, spectrogram calculation module 118. The system further comprises hardware for Doppler beam control 116 and a display monitor 124. The preferred embodiment of the system also comprises application-specific software modules: vessel detection deep learning (DL) module 114, spectrogram quality evaluation module 120, and vessel classification module 122 are implemented as software modules in the ultrasound scanner 110 hardware or on the PC side.

In a preferred embodiment, the image analysis system 100 as described above, is configured to execute the following procedure: an operator slowly moves the ultrasound probe 102 coupled to the tissue surface 108 and obtains one or more B-mode images, which represent a vessel or few vessels that could be an artery 106 or a vein 104. The scanning plane could be a transverse view (as illustrated in FIG. 1) or a longitudinal view. The B-mode imaging module outputs ultrasound structural image sequences to be used in the vessel detection DL module 114. The vessel detection module contains a deep learning network that is generated and trained by a deep learning training paradigm. The vessel detection module processes the B-mode images frame-by-frame through the trained deep learning algorithm, which could be any structure of deep learning such as convolutional neural network and predicts a bounding box for each detected vessel. The location and approximate size of the vessels and the parameters, including sample volume size and position for spectral Doppler gates, are assumed to be proportional to the predicted bounding box's center and size. The vessel detection predictions are superimposed on the B-mode image, which can be represented by the bounding box or an elliptical shape of proportional size, and shown on the display monitor 124. The predictions of the vessel detection DL module 114 are passed to the Doppler beam control 116. The Doppler beam control 116 receives the location where to place the Doppler beam and approximate size of the Doppler gates to set. The beam is directed into the vessel based on the DL prediction. The ultrasound scanner 110 receives radio frequency (RF) signals from the location and passes the signals to the spectrogram calculation module 118 to obtain time-frequency domain representation of the blood flow dynamics. The spectrogram calculation techniques are well known (such as fast Fourier transform, continuous wavelet transform, or parametric methods) and will not be described in detail in the embodiments. Optionally, a wall filter is used to remove low blood flow velocities, which are the result of tissue movement. The obtained spectrogram for the automatically detected vessel is passed to the spectrogram quality evaluation module 120, which decides if the spectrogram is suitable to proceed to classification.

The vessel classification module 122 could be implemented in two ways: by calculating a set of statistical quantities and inputting said statistical quantities into a trained ML algorithm, or by passing the obtained spectrogram as an image into a trained convolutional neural network. Statistical quantities such as the periodicity parameter of the envelope of the spectrogram are used in the first embodiment. The module formulates classifications of artery or vein, the result of which is overlaid with the B-mode image and displayed on the monitor.

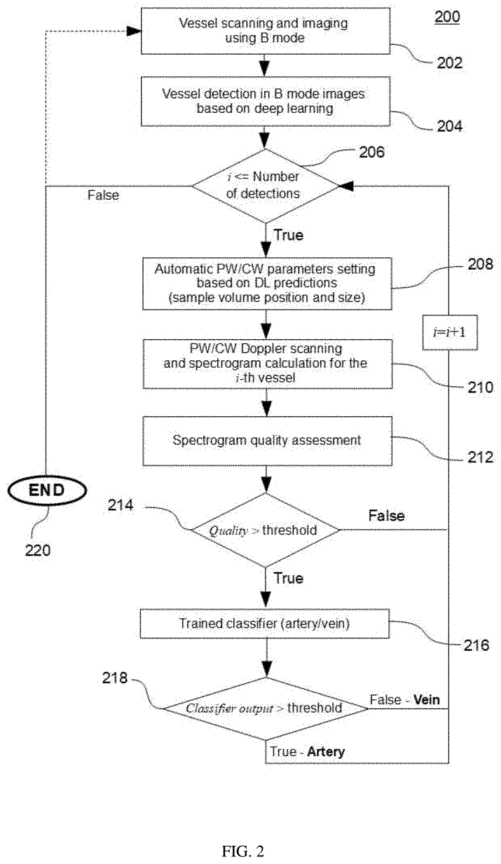

FIG. 2 presents a flowchart 200 of the method. First, a series of B-mode ultrasound images are acquired for a region containing blood vessels 202. A deep learning algorithm is trained to detect blood vessel(s) 204 in the structural B-mode images and outputs the location and size of a bounding box for each detected vessel. The bounding box dimensions and position are assumed to be proportional to vessel location and size. For each detected vessel 206, the following series of steps are executed. The parameters, PW gate location and size, are passed to the Doppler beam control module 208 and set for PW Doppler scanning of i-th vessel. The i-th vessel is scanned in PW mode with high pulse repetition frequency. The received RF signals are used to calculate a corresponding spectrogram representing blood flow velocities 210, and then the obtained spectrogram is assessed for quality 212. Several parameters, which estimate the proportion of blood flow related pixels to background noise are extracted, weighted, and combined. The combined signal-to-noise parameters are processed through the spectrogram quality assessment algorithm and compared with a quality threshold 214 obtained through a training procedure. If the quality of the spectrogram of i-th vessel is sufficient for quantitative analysis, the spectrogram is passed to the trained classifier 216. The outlined procedure is repeated for the next detected (i+1) vessel, and so on. The (i+1)th vessel is scanned by PW mode and evaluated in terms of spectrogram quality. If the spectrogram quality is sufficient for the analysis, then the spectrogram is passed to the trained classifier 216.

The trained classifier 216 could be implemented in two ways: by calculating statistical quantities and inputting the statistical quantities into a trained machine learning algorithm or by imputing the spectrogram image into a trained deep learning algorithm. The details of the preferred embodiments for classification are described in more detail below. If the output of the trained classifier 216 exceeds a predefined threshold 218, which was obtained through the training procedure, the vessel is classified as artery, otherwise it is classified as a vein. The steps of classification, 208-218 are repeated for each detected vessel. The procedure is concluded 220 when all the detected vessels are analysed and classified, and the procedure can be repeated for a new set of B-mode images.

FIG. 1 contains several modules which are inherent for state-of-the-art medical ultrasound imaging systems such as the B-mode imaging module 112 and spectrogram calculation module 118, and these modules will not be described in detail in the embodiments. For understanding, the B -mode imaging module provides the structural images and represents the magnitude of the reflections from the structures having different acoustic impedance. PW imaging are used for blood flow analysis and represent how blood flow velocities spectra are changing in time. The vessel is scanned with high pulse repetition frequency (order of kHz) to obtain sufficient sampling, then the Doppler frequency shift is estimated, and the spectrum of the Doppler signal is calculated.

The vessel detection DL module 114 is dedicated for use with structural B-mode images. The module utilizes deep learning principles, and the preferred embodiments use trained convolutional neural networks. In a preferred embodiment, deep learning networks that utilize fast architecture are used so that the computation time for vessel detection is commensurate with real-time brightness mode image processing frame rate, at least 50 frames/second, such as YOLO, Fast R-CNN, Faster R-CNN, or other comparatively fast architectures.

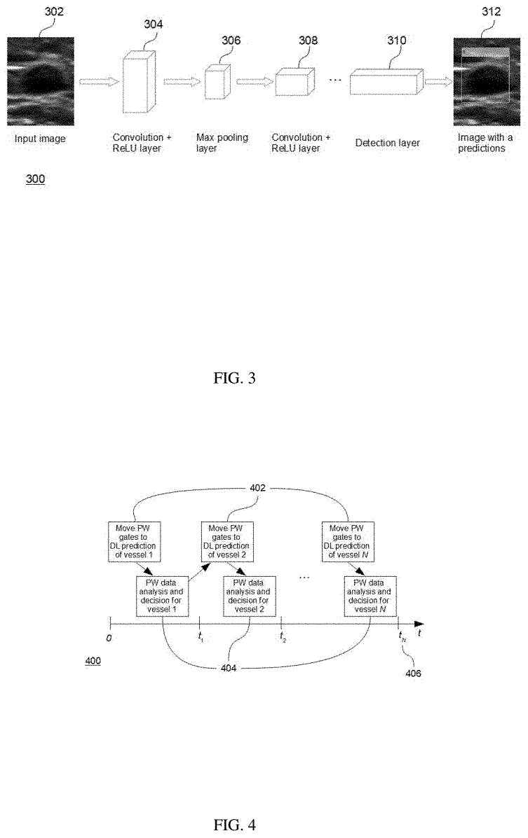

A sketch of the preferred convolutional network architecture with base components is shown in FIG. 3. The trained network 300 consists of 1) an input layer 302, to which the ultrasound image is passed; 2) N convolutional+Rectified Linear Unit (ReLU) layers 304, 308, wherein layers are repeated several times with different dimensions of input and output; 3) K max pooling layers 306 (in FIG. 3 one max pooling layer is shown, and the number of these layers depends on the selected architecture, which may contain more than one) and 4) the detection layer 310 which is individual for different neural network architectures (for example a fully connected layer) and will not be described in the embodiments in detail. The trained network 300 outputs the predictions 312 of the location and size of the bounding boxes for the object. The convolution and ReLU function layer 304 calculates convolutions between input image and the trained coefficients of the network ω. The convolutional layer is preferably expressed by the following formula:

Z=ω^{T}·X+b,

where Z is the output of convolutional layer, X is the input image matrix of I×J×K size, I is the number of image columns, J is the number of image rows, K is the number of channels, ω is the matrix of weighting coefficients, which could also be called L×L convolutional filter kernel, L is the size of a filter, and b is the bias coefficients vector, which is also obtained in the training phase. Multi-dimensional convolution is followed by the ReLU function. The ReLU function changes negative values of convolutional layer output to zeros and could be expressed as follows:

ReLU(Z)=max(0,Z).

A max pooling layer 306 is dedicated for down-sampling of the detection features in feature maps. It is realized by a maximum detector and selector in the predefined size window in feature maps. The convolution and ReLU function layer 308 is then repeated followed by a max pooling layer and so on until a prediction is made 312.

Training of the vessel detection deep learning network can be completed either offline or online using training data in a server-based database. For the offline training scheme, a representative database of B-mode image sequences and annotations must be collected. The annotations mean the bounding boxes of the vessels detected and outlined manually in B-mode images by an expert who visually evaluates the images. Optionally the vessels could be verified by using spectral Doppler to verify if the detected structure is a vessel. The collection of images and annotations are passed to the training procedure. Optimal weighing coefficients are obtained by using stochastic gradient descent (SGD) method or other techniques such as the Adam optimization algorithm. In the case of SGD, neural network weighing coefficients are updated by the following formula:

$${ω}_{i} = {ω}_{i - 1} - \frac{η}{n} {\sum }_{i = 1}^{n} ∇ {L}_{i} ( ω )$$

where η is a learning rate, i is the training iteration number, L is the loss function, and n is the number of observations.

The online training option requires a server-based database, which is connected to the ultrasound machine controlling PC (hereafter referred to as a workplace) to obtain new images and annotations performed by an expert; a picture archiving and communication system will serve for the purpose. The weighting coefficients of the neural network in such cases are updated with each new example received from the workplace. Continual training of the neural network produces a more reliable outcome.

The Doppler beam control 116 is dedicated for automatic adjustment of sample volume (Doppler gates) position and size, which are calculated based on the predictions of the vessel detection DL module 114. FIG. 4 illustrates a diagram for the spectral Doppler beam control and PW data analysis over time. The procedure includes both beam control 402 and spectral Doppler data analysis 404 for each detected vessel. The doppler beam is controlled by a specific set of delays for groups of elements that form a Doppler beam. The delays are calculated according to the detected position of a blood vessel, which is the centre of the predicted bounding box in the detection stage. The beam control and PW data analysis algorithm is sequential: when the beam is directed to the detected vessel, the calculated spectrogram is analysed 404 for quality control and for the vessel type classification (artery or vein). The scanning and analysis period 406 for one vessel should not exceed two t₁=2×heartbeats. When the classification for the first vessel is achieved, the beam is directed 402 to the next detected vessel, if there are more than one vessel detected, then the spectral Doppler data are analysed for the second vessel and so on.

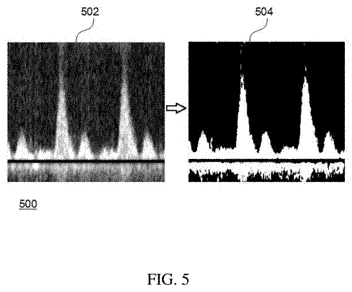

The spectrogram quality evaluation module 120 assesses whether the spectrogram is of sufficient quality for quantitative analysis. Venous flow is sometimes very weak and cannot be detected by spectral Doppler ultrasound especially if the imaging and Doppler scanning is performed in the transverse plane. The obtained spectrogram could be classified into two classes according to pixel intensities: blood flow information and background noise. For this purpose, the spectrogram 502 is binarized 504 to obtain a mask for blood flow related information extraction. The procedure of binarization is illustrated by 500 in FIG. 5. In general, the histogram of spectrogram intensities is bimodal, and the Otsu technique is used as it is the best mode to identify blood flow related pixels. The Otsu's algorithm finds the threshold that minimizes the intra-class variance defined as a weighted sum of variances of two classes:

σ_{w}²(t)=w₀(t)·σ₀²(t)+w₁(t)·σ₁²(t),

where w₀, w₁ are probabilities of the two classes 1 and 2 separated by a threshold t, and σ₀², σ₁² are variances of the classes.

In the next stage, the parameters of blood flow related pixel intensities are extracted. Two parameters of the proportion of blood flow related pixels in comparison to background are used: the ratio between the detected foreground pixels and a total number of pixels in a spectrogram, and the ratio between the sum of the intensities in the foreground and the sum of all intensities of the spectrogram. Finally, the parameters are combined into a vector and used for spectrogram classification into two classes: 1) sufficient quality and 2) insufficient quality. The optimal weights for the parameters are obtained through a training procedure. The parameters could be combined by using linear technique:

Y=w·X,

where Y is the output of a linear classifier and w is a weighting coefficients vector of a parameter X. Or by using non-linear classifiers such as support vector machines or others. Output of the classifier are compared to threshold values obtained through a training procedure. If the spectrogram is classified as of sufficient quality, the spectrogram passes to the vessel classification module 122. Spectrogram of insufficient quality cannot be used because the blood flow of veins is relatively weak and could be misidentified in a spectrogram with insufficient quality.

The vessel classification module 122 can be implemented by the following two embodiments. In the first embodiment, first mean velocity is calculated from the spectrogram then the spectrogram is parametrized. Next, four statistical quantities are calculated for blood flow characterization and classification:
1. Skewness of mean velocity vs. time dependence curve. In the case of arteries, the mean velocities are more positively skewed due to the presence of higher mean velocities in the distribution, meanwhile in the case of veins the mean velocities are distributed more symmetrically.
2. Presence of periodicity. The presence of periodicity is evaluated by calculating half of the windowed autocorrelation function of the extracted mean velocity curve. The function is calculated as follows:

$$R [ γ ] = {\sum }_{i = 0}^{N - Y} x [ i ] \cdot x [ j + γ ] ,$$

where γ is delay, 0≤y≤N, N is the number of samples of the mean velocity curve, x is the mean velocity value at a certain time instance. The obtained function is multiplied by a triangular window function in order to supress the peak at zero delay and to enhance peaks arising due to heart beat related pulsatility:

$$w [ n ] = 1 - \vert \frac{n - \frac{N}{2}}{\frac{N}{2}} \vert ,$$

where 0≤n≤N, N is the number of samples in the autocorrelation function. Finally, the presence of periodicity is evaluated by finding the maximum peak of the windowed autocorrelation function. The value of the peak serves as a statistical quantity for spectrogram characterization. A higher peak value indicates that there is a periodic pattern in the mean velocity curve, which is characteristic for arteries.
3. Skewness of the windowed half of autocorrelation function. In the case of arteries, the distribution of the function is positively skewed due to the presence of peaks, which represents the periodicity; meanwhile in the case of veins, the autocorrelation function is more symmetric.
4. Hjorth parameter of signal complexity:

$$C = \frac{M ( \frac{d x}{d t} )}{M ( x )} ,$$

where M is the mobility parameter, x is the mean velocity vs. time curve. Mobility is then calculated as follows:

$$M = \sqrt{\frac{var ( \frac{d x}{d t} )}{var ( x )}} ,$$

Where var is statistical variance. For mean velocity in veins, the signal complexity is lower while in the case of arteries, the mean velocity curve shape closely resembles a sine wave.

The statistical quantities are combined into a feature function and passed to the machine learning-based classifier, which determines if the scanned vessel belongs to an artery class or to a vein class. The trained classification algorithm could be a machine learning technique such as linear regression, non-linear, support vector machine classifier, or others.

In the second embodiment, the vessel classification module 122 is implemented by using deep learning principles, which evaluate the spectrogram directly using the principles of image recognition in a trained deep learning algorithm rather than evaluating the statistical quantities separately and combining each statistical quantity into a simplified feature function. In such case the spectrogram is passed into a trained convolutional neural network as an image and the network classifies the detected vessel to be an artery or a vein. The convolutional neural network for vessel classification architecture must be fast and the number of layers should not exceed 30. The feature extraction layers, including convolutional+ReLU, max pooling, are of a similar structure as shown in FIG. 3., but here, the final layer is dedicated for classification instead of detection. Implementation of the classification layer in principle is similar to the convolutional network used in vessel detection including a fully connected final layer. The network is trained by using spectrograms of previously classified arteries/veins. The exact class for training of the detected vessel type, artery or vein, are verified by applying a compression test by scanning probe. The test is performed in the following order: firstly, the vessel is detected by the first convolutional neural network in ultrasound image, secondly, the spectral Doppler scan is performed and spectrogram recorded, and finally the scanning performing technician applies gentle compression by scanning probe into the surface of tissue and if the vessel reacts to applied strain (deforms) that means that analysed vessel is vein, meanwhile arteries preserve their shape under compression due to relatively high blood flow pressure inside. The network could be trained online and offline, similarly to the vessel detection convolutional neural network.

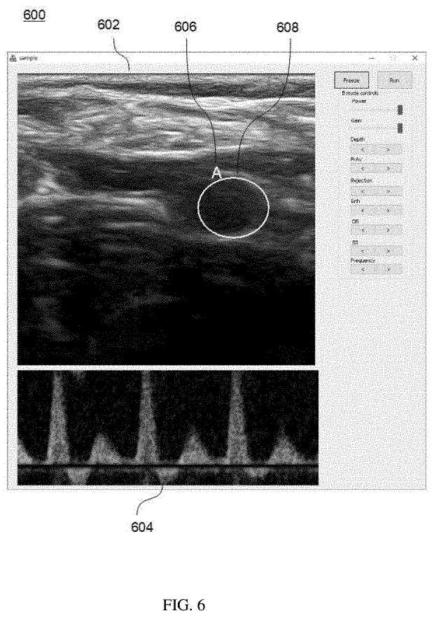

Finally, the results of the method are represented on the display monitor 124, typical on a PC or laptop monitor via a graphical user interface 600 (FIG. 6). The displayed data must contain: 1) B-mode ultrasound stream 602, 2) outlined detected vessels (for example by ellipse or rectangle) 608, 3) optionally Doppler spectrogram for visual evaluation 604 and 4) marking of detected vessel as artery or vein by, optionally: 1) by red contour for arteries, blue for veins, or 2) label with letters “A” or “V” close to the detected vessel 606, or 3) indicator in the GUI beside B-mode imaging window. FIG. 6 shows an example of the graphical user interface 600 which outputs the results of artery detection and identification.

1. A method for real-time image analysis of a series of brightness mode and doppler ultrasound images of a tissue sample, comprising:
detecting vessels from the series of brightness mode images using a deep learning algorithm that is trained for vessel detection and returning location and size of a bounding box of a detected vessel; and
further comprising for each detected vessel from the series of brightness mode images:
parameterizing for pulse wave Doppler gate placement using location and size of the bounding box of the detected vessel;
scanning the tissue sample using the Doppler gate parameterization and using the scanning data to produce a time-frequency domain Doppler spectrogram;
assessing the quality of the Doppler spectrogram using a first trained machine learning classifier algorithm and repeating the parameterization and scanning if the spectrogram quality is classified as insufficient;
passing the Doppler spectrogram of sufficient quality to a classification module;
classifying the vessel as an artery or a vein using a second trained machine learning classifier of the classification module; and
outputting the brightness mode image masked with an indication of vessel location and classification of the vessel.

2. The method of claim 1, wherein the deep learning algorithm that is trained for vessel detection is configured to process at least 50 brightness mode image frames per second.

3. The method of claim 2, wherein assessing the quality of the time-frequency domain Doppler spectrogram using a first trained machine learning classifier comprises:
classifying each pixel as either blood flow related data or noise based on pixel intensity;
minimizing an intra-class variance by evaluating a weighted sum of variances of the two classes;
extracting pixel intensities;
evaluating a first and second parameters for the proportion of blood flow related pixels in comparison to background, wherein the first parameter is a ratio between the blood flow related pixels and the total number of pixels in the spectrogram, and the second parameter is a ratio between a sum of the blood flow related pixel intensities and a sum of all pixel intensities of the spectrogram;
combining the first and second parameters into a feature vector; and
classifying the spectrogram as either of sufficient quality or of insufficient quality by evaluating the feature vector in a trained machine learning algorithm.

4. The method of claim 3, wherein classifying the vessel comprises:
parameterizing the Doppler spectrogram, wherein parameterizing comprises evaluation of statistical quantities: mean velocity from the time-frequency domain Doppler spectrogram, skewness of the mean velocity versus time curve, maximum peak of a windowed half of an autocorrelation function, skewness of the half of the autocorrelation function, and the Hjorth parameter of signal complexity; and combination of the statistical quantities into a feature function; and
classifying the vessel as an artery or a vein by evaluating the feature function in the second trained machine learning classifier.

5. The method of claim 3, wherein classifying the vessel comprises evaluating the time-frequency domain Doppler spectrogram in the second trained machine learning classifier, wherein the second trained machine learning classifier is a trained convolutional neural network.

6. The method of claim 3, further comprising displaying the Doppler spectrogram.

7. A system for real-time image analysis of a series of brightness mode and doppler ultrasound images of a tissue sample comprising an ultrasound probe and an ultrasound scanner, wherein the ultrasound scanner comprises a display monitor and one or more computer processors configured to execute one or more computer program products, the computer program products being tangibly embodied on a non-transitory computer-readable medium and comprising executable code for:
receiving a series of ultrasound signals at the one or more computer processors;
producing a series of brightness mode images from the series of ultrasound signals;
detecting vessels from the series of brightness mode images using a deep learning algorithm that is trained for vessel detection and returning location and size of a bounding box of a detected vessel; and
further comprising for each detected vessel from the series of brightness mode images:
parameterizing for pulse wave Doppler gate placement using location and size of the bounding box of the detected vessel;
scanning the tissue sample using the Doppler gate parameterization and using the scanning data to produce a time-frequency domain Doppler spectrogram;
assessing the quality of the Doppler spectrogram using a first trained machine learning classifier algorithm and repeating the parameterization and scanning if the spectrogram quality is classified as insufficient;
passing the Doppler spectrogram of sufficient quality to a classification module;
classifying the vessel as an artery or a vein using a second trained machine learning classifier of the classification module; and
outputting the brightness image masked with an indication of vessel location and classification of the vessel.

8. The system of claim 7, wherein the one or more computer processors are embedded in the ultrasound scanner and/or in a personal computer that is connected to the ultrasound scanner.

9. The system of claim 7, wherein the deep learning algorithm that is trained for vessel detection is configured to process at least 50 brightness mode image frames per second.

10. The system of claim 7, wherein assessing the quality of the time-frequency domain Doppler spectrogram using a first trained machine learning classifier comprises:
classifying each pixel as either blood flow related data or noise based on pixel intensity;
minimizing an intra-class variance by evaluating a weighted sum of variances of the two classes;
extracting pixel intensities;
evaluating two parameters for the proportion of blood flow related pixels in comparison to background, wherein a first parameter is a ratio between the blood flow related pixels and the total number of pixels in the spectrogram, and a second parameter is a ratio between a sum of the blood flow related pixel intensities and a sum of all pixel intensities of the spectrogram;
combining the first and second parameters into a feature vector; and
classifying the spectrogram as either of sufficient quality or of insufficient quality by evaluating the feature vector in a trained machine learning algorithm.

11. The system of claim 10, wherein classifying the vessel comprises:
parameterizing the Doppler spectrogram, wherein parameterizing comprises evaluation of statistical quantities: mean velocity from the time-frequency domain Doppler spectrogram, skewness of the mean velocity versus time curve, maximum peak of a windowed half of an autocorrelation function, skewness of the half of autocorrelation function, and the Hjorth parameter of signal complexity; and combination of the statistical quantities into a feature function; and
classifying the vessel as an artery or a vein by evaluating the feature function in the second trained machine learning classifier.

12. The system of claim 10, wherein classifying the vessel comprises evaluating the time-frequency domain Doppler spectrogram in the second trained machine learning classifier, wherein the second trained machine learning classifier is a trained convolutional neural network.

13. The system of claim 7, further comprising displaying the Doppler spectrogram.
